(19) United States
(12) Patent Application Publication
(10) Pub. No.: US 2026/0345583 A1
(43) Pub. Date: Nov. 5, 2026

(54) METHODS AND COMPOSITIONS FOR TREATING SLEEP APNEA

(71) Applicant: APNIMED, INC. (DELAWARE), Cambridge, MA (US)

(72) Inventors: Lawrence G. MILLER, Cambridge, MA (US); Luigi TARANTO-MONTEMURRO, Brookline, MA (US); Ronald FARKAS, Clarksville, MD (US); Yoshitaka ICHIKAWA, Laguna Niguel, CA (US)

(21) Appl. No.: 19/107,788

(22) PCT Filed: Aug. 30, 2023
(86) PCT No.: PCT/US2023/031509

§ 371 Date: Feb. 28, 2025

Related U.S. Application Data
(60) Provisional application No. 63/403,469, filed on Sep. 2, 2022.

(51) Int. Cl. A61K 31/4725; A61K 31/138; A61K 31/216; A61K 31/496; A61K 45/06

(52) U.S. Cl. CPC A61K 31/4725; A61K 31/138; A61K 31/216; A61K 31/496; A61K 45/06

(57) ABSTRACT

Methods of treating obstructive sleep apnea using an aldosterone synthase inhibitor, optionally in combination with a norepinephrine reuptake inhibitor or other active agent are disclosed herein, as well as related pharmaceutical compositions and therapeutic combinations.

### CROSS-REFERENCE TO RELATED APPLICATION

This application claims priority to and benefit of U.S. provisional patent application No. 63/403,469, filed Sep. 2, 2022, the entire contents of which are incorporated herein by reference.

### TECHNICAL FIELD

The present invention relates to methods of treating obstructive sleep apnea using an aldosterone synthase inhibitor, optionally in combination with a norepinephrine reuptake inhibitor or other active agent, as well as related pharmaceutical compositions and therapeutic combinations.

### BACKGROUND

Obstructive Sleep Apnea (OSA) is a common disorder caused by collapse of the pharyngeal airway during sleep. OSA can have serious health consequences.

### SUMMARY

One aspect of the present invention provides a method of treating a subject having a condition associated with pharyngeal airway collapse, the method comprising administering to a subject in need thereof an effective amount of baxdrostat, having the structure:

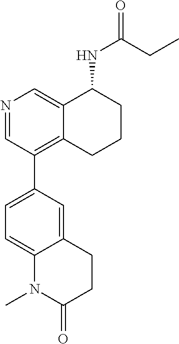 [SMILES: CCC(=O)N[C@@H]1CCCc2c(-c3ccc4c(c3)CCC(=O)N4C)cncc21]

or a pharmaceutically acceptable salt thereof.

Embodiments of this aspect of the invention may include one or more of the following optional features. In some embodiments, the method further comprises administering to the subject a norepinephrine reuptake inhibitor (NRI). In some embodiments, the NRI is a norepinephrine selective reuptake inhibitor (NSRI). In some embodiments, the NSRI is selected from the group consisting of amedalin, atomoxetine, CP-39,332, daledalin, edivoxetine, esreboxetine, lortalamine, nisoxetine, reboxetine, talopram, talsupram, tandamine, and viloxazine, or a pharmaceutically acceptable salt thereof. In some embodiments, the NRI is a norepinephrine non-selective reuptake inhibitor (NNRI) selected from the group consisting of amitriptiline, amoxapine, bupropion, ciclazindol, desipramine, desvenlafaxine, dexmethilphenidate, diethylpropion, doxepin, duloxetine, imipramine, levomilnacipran, manifaxine, maprotiline, methylphenidate, milnacipran, nefazodone, nortriptyline, phendimetrazine, phenmetrazine, protryptyline, radafaxine, tapentadol, teniloxazine, and venlafaxine, or a pharmaceutically acceptable salt thereof. In some embodiments, the NRI is selected from the group consisting of atomoxetine or a pharmaceutically acceptable salt thereof and reboxetine or a pharmaceutically acceptable salt thereof. In some embodiments, the NRI is atomoxetine or a pharmaceutically acceptable salt thereof. In some embodiments, the method further comprises administering to the subject a muscarinic receptor antagonist (MRA), for example, in combination with the baxdrostat and NRI. In some embodiments, the MRA is selected from the group consisting of atropine, propantheline, bethanechol, solifenacin, darifenacin, tolterodine, fesoterodine, trospium, and oxybutynin, or a pharmaceutically acceptable salt thereof. In some embodiments, the MRA is selected from the group consisting of anisotropine, benztropine, biperiden, clidinium, cycrimine, dicyclomine, diphemanil, diphenidol, ethopropazine, glycopyrrolate, hexocyclium, isopropamide, mepenzolate, methixene, methscopolamine, oxyphencyclimine, oxyphenonium, procyclidine, scopolamine, tridihexethyl, and trihexyphenidyl, or a pharmaceutically acceptable salt thereof. In some embodiments, the MRA is oxybutynin or a pharmaceutically acceptable salt thereof. In some embodiments, the MRA is (R)-oxybutynin or a pharmaceutically acceptable salt thereof. In some embodiments, the MRA is racemic oxybutynin or a pharmaceutically acceptable salt thereof. In some embodiments, the method further comprises administering to the subject a hypnotic, for example, in combination with baxdrostat and optionally with an NRI. In some embodiments, the hypnotic is selected from the group consisting of zolpidem, zopiclone, eszopiclone, trazodone, zaleplon, benzodiazepines, gabapentin, tiagabine, and sodium oxybate, or a pharmaceutically acceptable salt thereof. In some embodiments, the hypnotic is trazodone or a pharmaceutically acceptable salt thereof. In some embodiments, the baxdrostat or pharmaceutically acceptable salt thereof is administered at a dose of from about 5 to about 500 mg In some embodiments, the baxdrostat or pharmaceutically acceptable salt thereof is administered at a dose of from about 25 to about 250 mg. In some embodiments, the atomoxetine or pharmaceutically acceptable salt thereof is administered at a dose of from about 20 to about 200 mg. In some embodiments, the atomoxetine or pharmaceutically acceptable salt thereof is administered at a dose of from about 25 to about 100 mg. In some embodiments, the oxybutynin or pharmaceutically acceptable salt thereof is administered at a dose of from about 1 to about 15 mg. In some embodiments, the oxybutynin or pharmaceutically acceptable salt thereof is administered at a dose of from about 2 mg to about 10 mg. In some embodiments, the (R)-oxybutynin or pharmaceutically acceptable salt thereof is administered at a dose of from about 0.5 to about 10 mg. In some embodiments, the (R)-oxybutynin or pharmaceutically acceptable salt thereof is administered at a dose of from about 1 mg to about 5 mg. In some embodiments, the trazodone or pharmaceutically acceptable salt thereof is administered at a dose of from about 12.5 mg to about 200 mg. In some embodiments, baxdrostat or a pharmaceutically acceptable salt thereof is administered as a monotherapy. In some embodiments, the NRI and baxdrostat or a pharmaceutically acceptable salt thereof are administered in a single composition. In some embodiments, the NRI, MRA, and baxdrostat or a pharmaceutically acceptable salt thereof are administered in a single composition. In some embodiments, the hypnotic and baxdrostat or a pharmaceutically acceptable salt thereof are administered in a single composition. In some embodiments, the hypnotic, the NRI, and baxdrostat or a pharmaceutically acceptable salt thereof are administered in a single composition. In some embodiments, the single composition is an oral administration form. In some embodiments, the oral administration form is a syrup, pill, tablet, troche, capsule, or patch. In some embodiments, the condition associated with pharyngeal airway collapse is sleep apnea. In some embodiments, the condition associated with pharyngeal airway collapse is obstructive sleep apnea (OSA). In some embodiments, the condition associated with pharyngeal airway collapse is snoring. In some embodiments, the condition associated with pharyngeal airway collapse is simple snoring. In some embodiments, the subject is in a non-fully conscious state, such as sleep. In some embodiments, the subject has hypertension. In some embodiments, the subject has OSA with hypertension.

Also provided herein is baxdrostat or a pharmaceutically acceptable salt thereof for use in treating obstructive sleep apnea.

Also provided herein is baxdrostat or a pharmaceutically acceptable salt thereof for use in treating obstructive sleep apnea with hypertension.

Also provided herein is a therapeutic combination of baxdrostat or a pharmaceutically acceptable salt thereof and (a) a norepinephrine reuptake inhibitor (NRI), and optionally a muscarinic receptor antagonist (MRA), or (b) a hypnotic, and optionally a norepinephrine reuptake inhibitor (NRI), for use in treating obstructive sleep apnea.

Also provided herein is a therapeutic combination of baxdrostat or a pharmaceutically acceptable salt thereof and (a) a norepinephrine reuptake inhibitor (NRI), optionally with a muscarinic receptor antagonist (MRA), or (b) a hypnotic, optionally with a norepinephrine reuptake inhibitor (NRI), for use in treating obstructive sleep apnea with hypertension.

Another aspect of the invention provides a method of treating a subject having a condition associated with pharyngeal airway collapse, the method comprising administering to a subject in need thereof an effective amount of a Compound of Formula (A), having the structure:

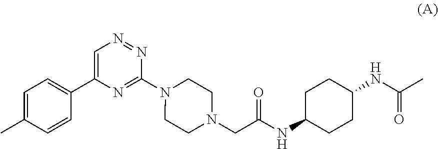 [SMILES: CC(=O)N[C@H]1CC[C@H](NC(=O)CN2CCN(c3nncc(-c4ccc(C)cc4)n3)CC2)CC1]

or a pharmaceutically acceptable salt thereof.

Embodiments of this aspect of the invention may include one or more of the following optional features. In some embodiments, the method further comprises administering to the subject a norepinephrine reuptake inhibitor (NRI). In some embodiments, the NRI is a norepinephrine selective reuptake inhibitor (NSRI). In some embodiments, the NSRI is selected from the group consisting of amedalin, atomoxetine, CP-39,332, daledalin, edivoxetine, esreboxetine, lortalamine, nisoxetine, reboxetine, talopram, talsupram, tandamine, and viloxazine, or a pharmaceutically acceptable salt thereof. In some embodiments, the NRI is a norepinephrine non-selective reuptake inhibitor (NNRI) selected from the group consisting of amitriptiline, amoxapine, bupropion, ciclazindol, desipramine, desvenlafaxine, dexmethilphenidate, diethylpropion, doxepin, duloxetine, imipramine, levomilnacipran, manifaxine, maprotiline, methylphenidate, milnacipran, nefazodone, nortriptyline, phendimetrazine, phenmetrazine, protryptyline, radafaxine, tapentadol, teniloxazine, and venlafaxine, or a pharmaceutically acceptable salt thereof. In some embodiments, the NRI is selected from the group consisting of atomoxetine or a pharmaceutically acceptable salt thereof and reboxetine or a pharmaceutically acceptable salt thereof. In some embodiments, the NRI is atomoxetine or a pharmaceutically acceptable salt thereof. In some embodiments, comprising administering to the subject a muscarinic receptor antagonist (MRA), for example, in combination with the Compound of Formula (A) and the NRI. In some embodiments, the MRA is selected from the group consisting of atropine, propantheline, bethanechol, solifenacin, darifenacin, tolterodine, fesoterodine, trospium, and oxybutynin, or a pharmaceutically acceptable salt thereof. In some embodiments, the MRA is selected from the group consisting of anisotropine, benztropine, biperiden, clidinium, cycrimine, dicyclomine, diphemanil, diphenidol, ethopropazine, glycopyrrolate, hexocyclium, isopropamide, mepenzolate, methixene, methscopolamine, oxyphencyclimine, oxyphenonium, procyclidine, scopolamine, tridihexethyl, and trihexyphenidyl, or a pharmaceutically acceptable salt thereof. In some embodiments, the MRA is oxybutynin or a pharmaceutically acceptable salt thereof. In some embodiments, the MRA is (R)-oxybutynin or a pharmaceutically acceptable salt thereof. In some embodiments, the MRA is racemic oxybutynin or a pharmaceutically acceptable salt thereof. In some embodiments, the method further comprises administering to the subject a hypnotic, for example, in combination with the Compound of Formula (A) and optionally an NRI. In some embodiments, the hypnotic is selected from the group consisting of zolpidem, zopiclone, eszopiclone, trazodone, zaleplon, benzodiazepines, gabapentin, tiagabine, and sodium oxybate, or a pharmaceutically acceptable salt thereof. In some embodiments, the hypnotic is trazodone or a pharmaceutically acceptable salt thereof. In some embodiments, the Compound of Formula (A) or pharmaceutically acceptable salt thereof is administered at a dose of from about 1 to about 500 mg. In some embodiments, the Compound of Formula (A) or pharmaceutically acceptable salt thereof is administered at a dose of from about 5 to about 200 mg. In some embodiments, the atomoxetine or pharmaceutically acceptable salt thereof is administered at a dose of from about 20 to about 200 mg. In some embodiments, the atomoxetine or pharmaceutically acceptable salt thereof is administered at a dose of from about 25 to about 100 mg. In some embodiments, the oxybutynin or pharmaceutically acceptable salt thereof is administered at a dose of from about 1 to about 15 mg. In some embodiments, the oxybutynin or pharmaceutically acceptable salt thereof is administered at a dose of from about 2 mg to about 10 mg. In some embodiments, the (R)-oxybutynin or pharmaceutically acceptable salt thereof is administered at a dose of from about 0.5 to about 10 mg. In some embodiments, the (R)-oxybutynin or pharmaceutically acceptable salt thereof is administered at a dose of from about 1 mg to about 5 mg. In some embodiments, the trazodone or pharmaceutically acceptable salt thereof is administered at a dose of from about 12.5 mg to about 200 mg. In some embodiments, the Compound of Formula (A) or a pharmaceutically acceptable salt thereof is administered as a monotherapy. In some embodiments, the NRI and the Compound of Formula (A) or a pharmaceutically acceptable salt thereof are administered in a single composition. In some embodiments, the NRI, MRA, and the Compound of Formula (A) or a pharmaceutically acceptable salt thereof are administered in a single composition. In some embodiments, the hypnotic and the Compound of Formula (A) or a pharmaceutically acceptable salt thereof are administered in a single composition. In some embodiments, the hypnotic, the NRI, and the Compound of Formula (A) or a pharmaceutically acceptable salt thereof are administered in a single composition. In some embodiments, the single composition is an oral administration form. In some embodiments, the oral administration form is a syrup, pill, tablet, troche, capsule, or patch. In some embodiments, the condition associated with pharyngeal airway collapse is sleep apnea. In some embodiments, the condition associated with pharyngeal airway collapse is obstructive sleep apnea (OSA). In some embodiments, the condition associated with pharyngeal airway collapse is snoring. In some embodiments, the condition associated with pharyngeal airway collapse is simple snoring. In some embodiments, the subject is in a non-fully conscious state, such as sleep. In some embodiments, the subject has hypertension. In some embodiments, the subject has OSA with hypertension.

Also provided herein is the Compound of Formula (A) or a pharmaceutically acceptable salt thereof for use in treating obstructive sleep apnea.

Also provided herein is the Compound of Formula (A) or a pharmaceutically acceptable salt thereof for use in treating obstructive sleep apnea with hypertension.

Also provided herein is a therapeutic combination of the Compound of Formula (A) or a pharmaceutically acceptable salt thereof and (a) a norepinephrine reuptake inhibitor (NRI), and optionally a muscarinic receptor antagonist (MRA), or (b) a hypnotic, and optionally a norepinephrine reuptake inhibitor (NRI), for use in treating obstructive sleep apnea.

Also provided herein is a therapeutic combination of the Compound of Formula (A) or a pharmaceutically acceptable salt thereof and (a) a norepinephrine reuptake inhibitor (NRI), optionally with a muscarinic receptor antagonist (MRA), or (b) a hypnotic, optionally with a norepinephrine reuptake inhibitor (NRI), for use in treating obstructive sleep apnea with hypertension.

Another aspect of the invention provides a pharmaceutical composition comprising baxdrostat or a pharmaceutically acceptable salt thereof, a norepinephinephrine reuptake inhibitor, and a pharmaceutically acceptable carrier or excipient. In some embodiments, the pharmaceutical composition further comprises a hypnotic. In some embodiments, the pharmaceutical composition further comprises a muscarinic receptor antagonist (MRA).

Another aspect of the invention provides a pharmaceutical composition comprising baxdrostat or a pharmaceutically acceptable salt thereof, a hypnotic, and a pharmaceutically acceptable carrier or excipient.

Another aspect of the invention provides a pharmaceutical composition comprising the Compound of Formula (A) or a pharmaceutically acceptable salt thereof, a norepinephinephrine reuptake inhibitor, and a pharmaceutically acceptable carrier or excipient. In some embodiments, the pharmaceutical composition further comprises a hypnotic. In some embodiments, the pharmaceutical composition further comprises a muscarinic receptor antagonist (MRA).

Another aspect of the invention provides a pharmaceutical composition comprising the Compound of Formula (A) or a pharmaceutically acceptable salt thereof, a hypnotic, and a pharmaceutically acceptable carrier or excipient.

Unless otherwise defined, all technical and scientific terms used herein have the same meaning as commonly understood by one of ordinary skill in the art to which this invention belongs. Methods and materials are described herein for use in the present invention; other suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and FIGURES, and from the claims.

### BRIEF DESCRIPTION OF THE DRAWINGS

The following FIGURES are provided by way of example and are not intended to limit the scope of the claimed invention.

FIG. 1 is a graphic illustration of an obstructive apnea. The top channel shows the electroencephalogram (EEG) pattern of sleep. The next channel represents airflow. The next three channels show ventilator effort by movements of the rib cage and abdomen and changes in esophageal pressure, all of which reflect a respiratory effort against an occluded upper airway. The last channel indicates oxyhemoglobin saturation.

### DETAILED DESCRIPTION

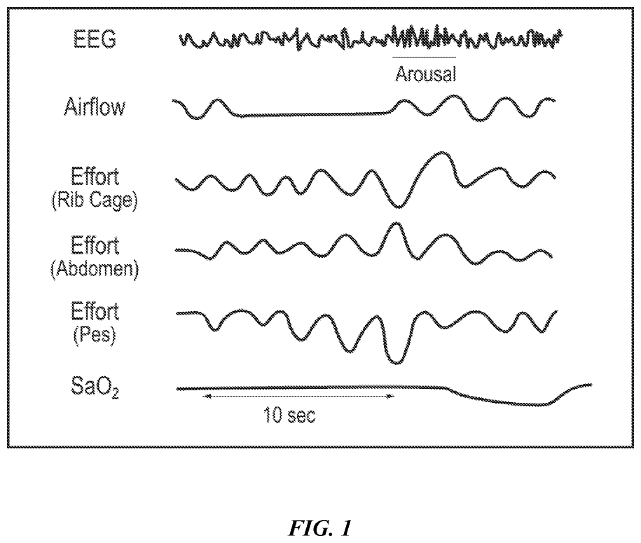

In humans, the pharyngeal airway region has no bone or cartilage support, and it is held open by muscles. When these muscles relax during sleep, the pharynx can collapse resulting in cessation of airflow. As shown in FIG. 1, ventilatory effort continues and increases in an attempt to overcome the obstruction, shown by an increase in esophageal pressure change. Rib cage and abdominal movements are in the opposite direction as a result of the diaphragm contracting against an occluded airway, forcing the abdominal wall to distend out and the chest wall to cave inward.

Increasing efforts to breathe lead to an arousal from sleep, visualisable on an EEG (FIG. 1), and result in opening of the airway and a resumption of normal breathing. The lack of airflow during the apnea also causes hypoxia, shown by a drop in oxyhemoglobin saturation (FIG. 1). Severity is generally measured using the apnea-hypopnea index (AHI), which is the combined average number of apneas (cessation of breathing for at least ten seconds) and hypopneas (reduced airflow and oxygen saturation) that occur per hour of sleep (Ruehland et al., The new AASM criteria for scoring hypopneas: Impact on the apnea hypopnea index. SLEEP 2009; 32(2):150-157).

FIG. 1 is a graphic illustration of an obstructive apnea. The top channel shows the electroencephalogram (EEG) pattern of sleep. The next channel represents airflow. The next three channels show ventilatory effort by movements of the rib cage and abdomen and changes in esophageal pressure, all of which reflect a respiratory effort against an occluded upper airway. The last channel indicates oxyhemoglobin saturation.

When a stringent definition of OSA is used (an AHI of >15 events per hour or AHI>5 events per hour with daytime sleepiness), the estimated prevalence is approximately 15 percent in males and 5 percent in females. An estimated 30 million individuals in the United States have OSA, of which approximately 6 million have been diagnosed. The prevalence of OSA in the United States appears to be increasing due to aging and increasing rates of obesity. OSA is associated with major comorbidities and economic costs, including: hypertension, diabetes, cardiovascular disease, motor vehicle accidents, workplace accidents, and fatigue/lost productivity. (Young et al., WMJ 2009; 108:246; Peppard et al., Am J Epidemiol 2013; 177:1006.)

The present leading treatment is continuous positive airway pressure (CPAP). CPAP is effective in virtually all patients, and approximately 85% of diagnosed patients are prescribed CPAP, but compliance is low. Patients find CPAP uncomfortable and often intolerable; at least 30% of patients (up to 80%) are regularly non-adherent and thus untreated (Weaver, Proc Am Thorac Soc. 2008 Feb. 15; 5(2): 173-178). Other treatment modalities with variable rates of success include oral appliances (10%) and surgery (5%), but neither is likely to be effective across the general population.

The search for medicines to activate pharyngeal muscles in sleeping humans has been discouraging; agents such as serotonin reuptake inhibitors, tricyclic antidepressants, and sedatives have all been tested in humans and shown to be ineffective at reducing OSA severity. See, e.g., Proia and Hudgel, Chest. 1991 August; 100(2):416-21; Brownell et al., N Engl J Med 1982, 307:1037-1042; Sangal et al., Sleep Med. 2008 July; 9(5):506-10. Epub 2007 Sep. 27; Marshall et al. p. 2008 June; 31(6):824-31; Eckert et al., Clin Sci (Lond). 2011 June; 120(12); 505-14; Taranto-Montemurro et al., Sleep. 2017 Feb. 1; 40(2).

In a recent study, a combination of atomoxetine and oxybutynin, referred to as “ato-oxy,” administered before bedtime has been shown to reduce OSA in patients with a wide range of severity. The ato-oxy combination, which was administered for one night, reduced the number of obstructive events, improved the overnight oxygen desaturation, and enhanced the genioglossus muscle activity in a group of unselected patients with OSA. The data collected in the proof-of-concept trial showed that it was possible to improve or abolish OSA using drugs with specific neurotransmitter profiles administered systemically. See Taranto-Montemurro, Luigi et al. “The Combination of Atomoxetine and Oxybutynin Greatly Reduces Obstructive Sleep Apnea Severity. A Randomized, Placebo-controlled, Double-Blind Crossover Trial.” American journal of respiratory and critical care medicine vol. 199, 10 (2019): 1267-1276.

There remains a need for further therapies for treating conditions associated with pharyngeal airway collapse such as sleep apnea.

### Methods of Treatment

The methods described herein include methods for the treatment of disorders associated with pharyngeal airway muscle collapse during sleep. In some embodiments, the disorder is sleep apnea (e.g., obstructive sleep apnea (OSA)) or snoring (e.g., simple snoring). In some embodiments, the subject has hypertension or OSA with hypertension. Generally, the methods include administering a therapeutically effective amount of an aldosterone synthase inhibitor selected from baxdrostat or a Compound of Formula (A), or pharmaceutically acceptable salts thereof, optionally with other active agent(s), to a subject who is in need of, or who has been determined to be in need of, such treatment.

As used in this context, to “treat” means to ameliorate at least one symptom of the disorder associated with pharyngeal airway collapse. Often, pharyngeal airway collapse during sleep results in snoring and/or an interruption in breathing (apnea or hypopnea), arousal from sleep, and reduced oxygenation (hypoxemia); thus, a treatment can result in a reduction in snoring, apneas/hypopneas, sleep fragmentation, and hypoxemia. Administration of a therapeutically effective amount of a compound described herein for the treatment of a subject with OSA may result in decreased AHI. Measurement of OSA disease and symptoms may be, for example, by polysomnography (PSG).

In general, an “effective amount” of a compound refers to an amount sufficient to elicit the desired biological response, e.g., to treat a condition associated with pharyngeal airway collapse, e.g., to treat sleep apnea or snoring. As will be appreciated by those of ordinary skill in this art, the effective amount of a compound of the invention may vary depending on such factors as the desired biological endpoint, the pharmacokinetics of the compound, the disease being treated, the mode of administration, and the age, weight, health, and condition of the subject. An effective amount encompasses therapeutic and prophylactic treatment.

An effective amount can be administered in one or more administrations, applications or dosages. The compositions can be administered from one or more times per day to one or more times per week; including once every other day. In some embodiments, the compositions are administered daily. In some embodiments, the compositions are administered daily before sleep time, e.g., immediately before sleep time or 15-60 minutes before sleep time. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, the terms “subject” and “patient” are used interchangeably. The terms “subject” and “patient” refer to an animal (e.g., a bird such as a chicken, quail or turkey, or a mammal), specifically a “mammal” including a non-primate (e.g., a cow, pig, horse, sheep, rabbit, guinea pig, rat, cat, dog, and mouse) and a primate (e.g., a monkey, chimpanzee and a human), and more specifically a human. In one embodiment, the subject is a non-human animal such as a farm animal (e.g., a horse, cow, pig or sheep), or a pet (e.g., a dog, cat, guinea pig or rabbit). In a preferred embodiment, the subject is a human.

As used herein, “pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

As used herein, the term “unit dosage form” is defined to refer to the form in which the compound is administered to a subject. Specifically, the unit dosage form can be, for example, a pill, capsule, or tablet. In some embodiments, the unit dosage form is a capsule.

As used herein, “solid dosage form” means a pharmaceutical dose(s) in solid form, e.g. tablets, capsules, granules, powders, sachets, reconstitutable powders, dry powder inhalers and chewables.

For the compounds disclosed herein, single stereochemical isomers, as well as enantiomers, diastereomers, cis/trans conformation isomers, and rotational isomers, and racemic and non-racemic mixtures thereof, are within the scope of the invention. Unless otherwise indicated, all tautomeric forms of the compounds disclosed herein are within the scope of the invention.

Baxdrostat and methods of its synthesis are known in the art. See U.S. Pat. No. 9,353,081, the entire contents of which are incorporated herein by reference. Baxdrostat is an aldosterone synthase inhibitor. The structure of baxdrostat is shown below.

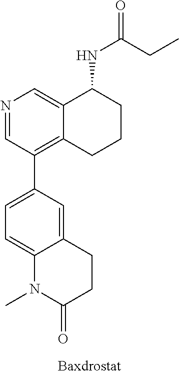 [SMILES: CCC(=O)N[C@@H]1CCCc2c(-c3ccc4c(c3)CCC(=O)N4C)cncc21]

The Compound of Formula (A) and methods of its synthesis are known in the art. See U.S. Pat. Nos. 10,029,993, 11,339,135, and WIPO Publication No. WO 2022/093714 A1, the entire contents of each of which are incorporated herein by reference. The Compound of Formula (A) is an aldosterone synthase inhibitor. The Compound of Formula (A) is shown below.

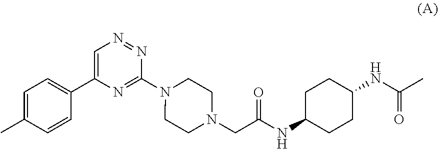 [SMILES: CC(=O)N[C@H]1CC[C@H](NC(=O)CN2CCN(c3nncc(-c4ccc(C)cc4)n3)CC2)CC1]

In some embodiments, the Compound of Formula (A) is in the form of a free base. In some embodiments, the Compound of Formula (A) is in the form of a pharmaceutically acceptable salt. In some embodiments, the Compound of Formula (A) is in the form of a monohydrobromide salt.

Atomoxetine is the generic name of the pharmaceutical substance with the chemical name (−)-N-Methyl-3-phenyl-3-(o-tolyloxy)-propylamine, and its pharmaceutical salts. Atomoxetine is the R(−) isomer as determined by x-ray diffraction. In some embodiments, atomoxetine may be atomoxetine hydrochloride.

Oxybutynin is the generic name for the pharmaceutical substance with the chemical name 4-diethylamino-2-butynylphenylcyclohexylglycolate or 4-(diethylamino)but-2-ynyl 2-cyclohexyl-2-hydroxy-2-phenylacetate, and its pharmaceutically acceptable salts. In various embodiments, oxybutynin may be a racemic mixture of R- and S-enantiomers, or an isolated enantiomer, e.g., the R-enantiomer. In various embodiments, oxybutynin may be oxybutynin chloride or (R)-oxybutynin chloride.

In some embodiments, baxdrostat or a pharmaceutically acceptable salt thereof can be administered via oral administration at a daily dosage of about 0.1 mg to 20 mg per kg body weight, preferably about 0.5 mg to 4 mg per kg body weight (e.g. about 300 mg per person), divided into preferably 1-3 individual doses, which can consist, for example, of the same amounts. For example, a daily dosage may be from about 1 mg to about 2000 mg, preferably about 5 mg to about 500 mg, or about 25 mg to about 250 mg. It will, however, be clear that the upper limit given herein can be exceeded when this is shown to be indicated.

In some embodiments, the Compound of Formula (A) or a pharmaceutically acceptable salt thereof is administered at a dose of from about 0.001 mg to about 1,000 mg. In some embodiments, the dose is from about 0.01 mg to about 900 mg. In some embodiments, the dose is from about 0.1 mg to about 800 mg. In some embodiments, the dose is from about 1 mg to about 700 mg. In some embodiments, the dose is from about 1 mg to about 600 mg. In some embodiments, the dose is from about 1 mg to about 500 mg. In some embodiments, the dose is from about 1 mg to about 400 mg. In some embodiments, the Compound of Formula (A) is dosed according to one of the following:
(i) between 5 mg and 100 mg administered orally twice a day, e.g., 12 hours apart;
(ii) between 10 mg and 50 mg administered orally twice a day, e.g., 12 hours apart;
(iii) between 5 mg and 100 mg administered orally once a day;
(iv) between 10 mg and 50 mg administered orally once a day.
(v) 12.5 mg administered orally twice a day, e.g., 12 hours apart;
(vi) 25 mg administered orally twice a day, e.g., 12 hours apart;
(vii) 12.5 mg administered orally once a day;
(viii) 50 mg administered orally once a day; or
(ix) 100 mg administered orally once a day.

In some embodiments, the methods include administering a dose of from about 20 mg to about 200 mg of atomoxetine or a pharmaceutically acceptable salt thereof (or a dose equivalent of another NRI). In some embodiments, the dose of atomoxetine or a pharmaceutically acceptable salt thereof is from about 25 mg to about 100 mg. In some embodiments, the dose of atomoxetine or pharmaceutically acceptable salt thereof is from about 40 mg to about 80 mg. In some embodiments, the dose of atomoxetine or pharmaceutically acceptable salt thereof is from about 20 mg to about 50 mg. In some embodiments, the dose of atomoxetine or a pharmaceutically acceptable salt thereof is from about 50 mg to about 100 mg. In some embodiments, the dose of atomoxetine or pharmaceutically acceptable salt thereof is about 40 mg. In some embodiments, the dose of atomoxetine or pharmaceutically acceptable salt thereof is about 80 mg.

In methods comprising administration of oxybutynin or (R)-oxybutynin or a pharmaceutically acceptable salt thereof (or another MRA), the dose of oxybutynin or (R)-oxybutynin or pharmaceutically acceptable salt thereof may be from about 1 mg to about 25 mg (or a dose equivalent thereof of another MRA), or in some embodiments, from about 2 mg to about 15 mg. In some embodiments, the dose of oxybutynin or pharmaceutically acceptable salt thereof is from about 2.5 mg to about 10 mg, e.g., 5 mg. In some embodiments, the dose of (R)-oxybutynin or pharmaceutically acceptable salt thereof is from about 1 mg to about 5 mg, e.g., 2.5 mg. In some embodiments, the dose of oxybutynin or (R)-oxybutynin or pharmaceutically acceptable salt thereof is from about 1 mg to about 10 mg.

### Pharmaceutical Compositions

Also provided herein are pharmaceutical compositions comprising an aldosterone synthase inhibitor selected from baxdrostat or a Compound of Formula (A), or pharmaceutically acceptable salts thereof, as an active ingredient. In some embodiments, the pharmaceutical composition further comprises one or more additional active ingredients selected from an NRI, and MRA, and a hypnotic. The active ingredients can be in a single composition or in separate compositions. In certain embodiments, the pharmaceutical compositions include atomoxetine or a pharmaceutically acceptable salt thereof and optionally oxybutynin (e.g., (R)-oxybutynin) or a pharmaceutically acceptable salt thereof, as active ingredients. In certain embodiments, the pharmaceutical compositions include trazodone or a pharmaceutically acceptable salt thereof and optionally atomoxetine or a pharmaceutically acceptable salt thereof, as active ingredients.

Exemplary norepinephrine reuptake inhibitors (NRIs) include the selective NRIs, e.g., amedalin (UK-3540-1), atomoxetine (Strattera), CP-39,332, daledalin (UK-3557-15), edivoxetine (LY-2216684), esreboxetine, lortalamine (LM-1404), nisoxetine (LY-94,939), reboxetine (Edronax, Vestra), talopram (Lu 3-010), talsupram (Lu 5-005), tandamine (AY-23,946), viloxazine (Vivalan); and the non-selective NRIs, e.g., amitriptiline, amoxapine, bupropion, ciclazindol, desipramine, desvenlafaxine, dexmethilphenidate, diethylpropion, doxepin, duloxetine, imipramine, levomilnacipran, manifaxine (GW-320,659), maprotiline, methylphenidate, milnacipran, nefazodone, nortriptyline, phendimetrazine, phenmetrazine, protryptyline, radafaxine (GW-353,162), tapentadol (Nucynta), teniloxazine (Lucelan, Metatone) and venlafaxine; and pharmaceutically acceptable salts thereof.

In some embodiments, the NRI is atomoxetine or a pharmaceutically acceptable salt thereof.

Exemplary muscarinic receptor antagonists (MRAs) include atropine, propantheline, bethanechol, solifenacin, darifenacin, tolterodine, fesoterodine, trospium, and oxybutynin, and pharmaceutically acceptable salts thereof, which have activity on the M2 receptor. Other exemplary antimuscarinics include anisotropine, benztropine, biperiden, clidinium, cycrimine, dicyclomine, diphemanil, diphenidol, ethopropazine, glycopyrrolate, hexocyclium, isopropamide, mepenzolate, methixene, methscopolamine, oxyphencyclimine, oxyphenonium, procyclidine, scopolamine, tridihexethyl, and trihexyphenidyl, and pharmaceutically acceptable salts thereof.

In some embodiments, the muscarinic receptor antagonist is oxybutynin or a pharmaceutically acceptable salt thereof. In some embodiments the oxybutynin is racemic oxybutynin or (R)-oxybutynin, or a pharmaceutically acceptable salt thereof. As used herein, (R)-oxybutynin refers to the (R)-oxybutynin stereoisomer substantially free of other stereoisomers of oxybutynin. In some embodiments, the muscarinic receptor antagonist is fesoterodine.

Exemplary hypnotics include zolpidem, zopiclone, eszopiclone, trazodone, zaleplon, benzodiazepines, gabapentin, tiagabine, and sodium oxybate, and pharmaceutically acceptable salts thereof.

In some embodiments, the hypnotic is trazodone or a pharmaceutically acceptable salt thereof.

Pharmaceutical compositions typically include a pharmaceutically acceptable carrier. As used herein the language “pharmaceutically acceptable carrier” includes saline, solvents, dispersion media, diluents, fillers, coatings, antibacterial and antifungal agents, isotonic and absorption delaying agents, and the like, compatible with pharmaceutical administration.

The active ingredients for use in the present invention may be provided as a free base or as pharmaceutically acceptable salts. For example, in some embodiments, oxybutynin is oxybutynin chloride. In some embodiments, (R)-oxybutynin is (R)-oxybutynin chloride. In some embodiments, racemic oxybutynin is racemic oxybutynin chloride. In some embodiments, atomoxetine is atomoxetine hydrochloride.

Pharmaceutical compositions are typically formulated to be compatible with its intended route of administration. Examples of routes of administration include systemic oral or transdermal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, oral compositions generally include an inert diluent or an edible carrier. For the purpose of oral therapeutic administration, the active compound(s) can be incorporated with excipients and used in the form of pills, tablets, troches, or capsules, e.g., gelatin capsules. Oral compositions can also be prepared using a fluid carrier. In some embodiments, a composition according to the present invention may be a unit dosage form. In some embodiments, a composition according to the present invention may be a solid dosage form, e.g., a tablet or capsule.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Systemic administration of the compounds as described herein can also be by transdermal means, e.g., using a patch, gel, or lotion, to be applied to the skin. For transdermal administration, penetrants appropriate to the permeation of the epidermal barrier can be used in the formulation. Such penetrants are generally known in the art. For example, for transdermal administration, the active compounds can formulated into ointments, salves, gels, or creams as generally known in the art. The gel and/or lotion can be provided in individual sachets, or via a metered-dose pump that is applied daily; see, e.g., Cohn et al., Ther Adv Urol. 2016 April; 8(2): 83-90.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration or use in a method described herein.

In some embodiments, the pharmaceutical composition is for use in treating a condition associated with pharyngeal airway collapse. In some embodiments, the condition is sleep apnea (e.g., OSA) or snoring (e.g., simple snoring).

### Kits and Combinations

Also provided herein is a kit comprising an aldosterone synthase inhibitor selected from baxdrostat or the Compound of Formula (A) and one or more additional active ingredients selected from an NRI, an MRA, and a hypnotic. For example, the kit may comprise separate pharmaceutical compositions with each composition having a single active ingredient. The kits can be used for treating a subject having a condition associated with pharyngeal airway collapse. Various embodiments of kits will be apparent from the detailed description provided herein.

Also provided herein is an aldosterone synthase inhibitor selected from baxdrostat or the Compound of Formula (A) and one or more additional active ingredients selected from an NRI, an MRA, and a hypnotic, for use in treating a subject having a condition associated with pharyngeal airway collapse. Further provided herein is a therapeutic combination of an aldosterone synthase inhibitor selected from baxdrostat or the Compound of Formula (A) and one or more additional active ingredients selected from an NRI, an MRA, and a hypnotic, for use in treating a subject having a condition associated with pharyngeal airway collapse. Various embodiments of combinations and therapeutic combinations will be apparent from the detailed description provided herein. In certain embodiments of the kits, combinations, and therapeutic combinations of the present invention, the NRI, if present, is atomoxetine or a pharmaceutically acceptable salt thereof, the hypnotic, if present, is trazodone or a pharmaceutically acceptable salt thereof, and the MRA, if present, is oxybutynin (e.g., (R)-oxybutynin) or a pharmaceutically acceptable salt thereof.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating a subject having a condition associated with pharyngeal airway collapse, the method comprising administering to a subject in need thereof an effective amount of baxdrostat: 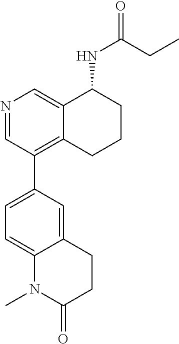 [SMILES: CCC(=O)N[C@@H]1CCCc2c(-c3ccc4c(c3)CCC(=O)N4C)cncc21]
or a pharmaceutically acceptable salt thereof.

2. The method of claim 1, further comprising administering to the subject a norepinephrine reuptake inhibitor (NRI).

3. The method of claim 2, wherein the NRI is a norepinephrine selective reuptake inhibitor (NSRI).

4. The method of claim 3, wherein the NSRI is selected from the group consisting of amedalin, atomoxetine, CP-39,332, daledalin, edivoxetine, esreboxetine, lortalamine, nisoxetine, reboxetine, talopram, talsupram, tandamine, and viloxazine, or a pharmaceutically acceptable salt thereof.

5. The method of claim 2, wherein the NRI is a norepinephrine non-selective reuptake inhibitor (NNRI) selected from the group consisting of amitriptiline, amoxapine, bupropion, ciclazindol, desipramine, desvenlafaxine, dexmethilphenidate, diethylpropion, doxepin, duloxetine, imipramine, levomilnacipran, manifaxine, maprotiline, methylphenidate, milnacipran, nefazodone, nortriptyline, phendimetrazine, phenmetrazine, protryptyline, radafaxine, tapentadol, teniloxazine, and venlafaxine, or a pharmaceutically acceptable salt thereof.

6. The method of claim 2, wherein the NRI is selected from the group consisting of atomoxetine or a pharmaceutically acceptable salt thereof and reboxetine or a pharmaceutically acceptable salt thereof.

7. The method of claim 6, wherein the NRI is atomoxetine or a pharmaceutically acceptable salt thereof.

8. The method of any one of claims 2-7, further comprising administering to the subject a muscarinic receptor antagonist (MRA).

9. The method of claim 8, wherein the MRA is selected from the group consisting of atropine, propantheline, bethanechol, solifenacin, darifenacin, tolterodine, fesoterodine, trospium, and oxybutynin, or a pharmaceutically acceptable salt thereof.

10. The method of any claim 8, wherein the MRA is selected from the group consisting of anisotropine, benztropine, biperiden, clidinium, cycrimine, dicyclomine, diphemanil, diphenidol, ethopropazine, glycopyrrolate, hexocyclium, isopropamide, mepenzolate, methixene, methscopolamine, oxyphencyclimine, oxyphenonium, procyclidine, scopolamine, tridihexethyl, and trihexyphenidyl, or a pharmaceutically acceptable salt thereof.

11. The method of claim 9, wherein the MRA is oxybutynin or a pharmaceutically acceptable salt thereof.

12. The method of claim 11, wherein the MRA is (R)-oxybutynin or a pharmaceutically acceptable salt thereof.

13. The method of claim 11, wherein the MRA is racemic oxybutynin or a pharmaceutically acceptable salt thereof.

14. The method of any one of claims 1-7, further comprising administering to the subject a hypnotic.

15. The method of claim 14, wherein the hypnotic is selected from the group consisting of zolpidem, zopiclone, eszopiclone, trazodone, zaleplon, benzodiazepines, gabapentin, tiagabine, and sodium oxybate, or a pharmaceutically acceptable salt thereof.

16. The method of claim 15, wherein the hypnotic is trazodone or a pharmaceutically acceptable salt thereof.

17. The method of any one of claims 1-16, wherein the baxdrostat or pharmaceutically acceptable salt thereof is administered at a dose of from about 5 to about 500 mg

18. The method of claim 17, wherein the baxdrostat or pharmaceutically acceptable salt thereof is administered at a dose of from about 25 to about 250 mg.

19. The method of any one of claims 1-18, wherein the atomoxetine or pharmaceutically acceptable salt thereof is administered at a dose of from about 20 to about 200 mg.

20. The method of claim 19, wherein the atomoxetine or pharmaceutically acceptable salt thereof is administered at a dose of from about 25 to about 100 mg.

21. The method of any one of claims 8-13, wherein the oxybutynin or pharmaceutically acceptable salt thereof is administered at a dose of from about 1 to about 15 mg.

22. The method of claim 21, wherein the oxybutynin or pharmaceutically acceptable salt thereof is administered at a dose of from about 2 mg to about 10 mg.

23. The method of any one of claims 8-12, wherein the (R)-oxybutynin or pharmaceutically acceptable salt thereof is administered at a dose of from about 0.5 to about 10 mg.

24. The method of claim 23, wherein the (R)-oxybutynin or pharmaceutically acceptable salt thereof is administered at a dose of from about 1 mg to about 5 mg.

25. The method of any one of claims 14-16, wherein the trazodone or pharmaceutically acceptable salt thereof is administered at a dose of from about 12.5 mg to about 200 mg.

26. The method of claim 1, 17 or 18 wherein baxdrostat or a pharmaceutically acceptable salt thereof is administered as a monotherapy.

27. The method of any one of claims 2-25, wherein the NRI and baxdrostat or a pharmaceutically acceptable salt thereof are administered in a single composition.

28. The method of any one of claims 8-13, wherein the NRI, MRA, and baxdrostat or a pharmaceutically acceptable salt thereof are administered in a single composition.

29. The method of any one of claims 14-16, wherein the hypnotic and baxdrostat or a pharmaceutically acceptable salt thereof are administered in a single composition.

30. The method of any one of claims 14-16, wherein the hypnotic, the NRI, and baxdrostat or a pharmaceutically acceptable salt thereof are administered in a single composition.

31. The method of any one of claims 27-30, wherein the single composition is an oral administration form.

32. The method of claim 31, wherein the oral administration form is a syrup, pill, tablet, troche, capsule, or patch.

33. The method of any one of claims 1-32, wherein the condition associated with pharyngeal airway collapse is sleep apnea.

34. The method of claim 33, wherein the condition associated with pharyngeal airway collapse is obstructive sleep apnea (OSA).

35. The method of any one of claims 1-32, wherein the condition associated with pharyngeal airway collapse is snoring.

36. The method of claim 35, wherein the condition associated with pharyngeal airway collapse is simple snoring.

37. The method of any one of claims 1-36, wherein the subject is in a non-fully conscious state, such as sleep.

38. The method of any one of claims 1-37, wherein the subject has hypertension.

39. The method of claim 38, wherein the subject has OSA with hypertension.

40. Baxdrostat or a pharmaceutically acceptable salt thereof for use in treating obstructive sleep apnea.

41. Baxdrostat or a pharmaceutically acceptable salt thereof for use in treating obstructive sleep apnea with hypertension.

42. A therapeutic combination of baxdrostat or a pharmaceutically acceptable salt thereof and (a) a norepinephrine reuptake inhibitor (NRI), and optionally a muscarinic receptor antagonist (MRA), or (b) a hypnotic, and optionally a norepinephrine reuptake inhibitor (NRI), for use in treating obstructive sleep apnea.

43. A therapeutic combination of baxdrostat or a pharmaceutically acceptable salt thereof and (a) a norepinephrine reuptake inhibitor (NRI), optionally with a muscarinic receptor antagonist (MRA), or (b) a hypnotic, optionally with a norepinephrine reuptake inhibitor (NRI), for use in treating obstructive sleep apnea with hypertension.

44. A method of treating a subject having a condition associated with pharyngeal airway collapse, the method comprising administering to a subject in need thereof an effective amount of the Compound of Formula (A): 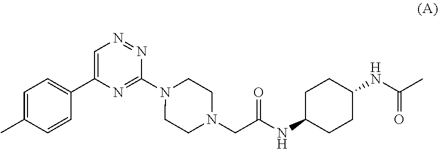 [SMILES: CC(=O)N[C@H]1CC[C@H](NC(=O)CN2CCN(c3nncc(-c4ccc(C)cc4)n3)CC2)CC1]
or a pharmaceutically acceptable salt thereof.

45. The method of claim 44, further comprising administering to the subject a norepinephrine reuptake inhibitor (NRI).

46. The method of claim 45, wherein the NRI is a norepinephrine selective reuptake inhibitor (NSRI).

47. The method of claim 46, wherein the NSRI is selected from the group consisting of amedalin, atomoxetine, CP-39,332, daledalin, edivoxetine, esreboxetine, lortalamine, nisoxetine, reboxetine, talopram, talsupram, tandamine, and viloxazine, or a pharmaceutically acceptable salt thereof.

48. The method of claim 45, wherein the NRI is a norepinephrine non-selective reuptake inhibitor (NNRI) selected from the group consisting of amitriptiline, amoxapine, bupropion, ciclazindol, desipramine, desvenlafaxine, dexmethilphenidate, diethylpropion, doxepin, duloxetine, imipramine, levomilnacipran, manifaxine, maprotiline, methylphenidate, milnacipran, nefazodone, nortriptyline, phendimetrazine, phenmetrazine, protryptyline, radafaxine, tapentadol, teniloxazine, and venlafaxine, or a pharmaceutically acceptable salt thereof.

49. The method of claim 45, wherein the NRI is selected from the group consisting of atomoxetine or a pharmaceutically acceptable salt thereof and reboxetine or a pharmaceutically acceptable salt thereof.

50. The method of claim 49, wherein the NRI is atomoxetine or a pharmaceutically acceptable salt thereof.

51. The method of any one of claims 45-50, further comprising administering to the subject a muscarinic receptor antagonist (MRA).

52. The method of claim 51, wherein the MRA is selected from the group consisting of atropine, propantheline, bethanechol, solifenacin, darifenacin, tolterodine, fesoterodine, trospium, and oxybutynin, or a pharmaceutically acceptable salt thereof.

53. The method of any claim 51, wherein the MRA is selected from the group consisting of anisotropine, benztropine, biperiden, clidinium, cycrimine, dicyclomine, diphemanil, diphenidol, ethopropazine, glycopyrrolate, hexocyclium, isopropamide, mepenzolate, methixene, methscopolamine, oxyphencyclimine, oxyphenonium, procyclidine, scopolamine, tridihexethyl, and trihexyphenidyl, or a pharmaceutically acceptable salt thereof.

54. The method of claim 52, wherein the MRA is oxybutynin or a pharmaceutically acceptable salt thereof.

55. The method of claim 54, wherein the MRA is (R)-oxybutynin or a pharmaceutically acceptable salt thereof.

56. The method of claim 54, wherein the MRA is racemic oxybutynin or a pharmaceutically acceptable salt thereof.

57. The method of any one of claims 44-50, further comprising administering to the subject a hypnotic.

58. The method of claim 57, wherein the hypnotic is selected from the group consisting of zolpidem, zopiclone, eszopiclone, trazodone, zaleplon, benzodiazepines, gabapentin, tiagabine, and sodium oxybate, or a pharmaceutically acceptable salt thereof.

59. The method of claim 58, wherein the hypnotic is trazodone or a pharmaceutically acceptable salt thereof.

60. The method of any one of claims 44-59, wherein the Compound of Formula (A) or pharmaceutically acceptable salt thereof is administered at a dose of from about 1 to about 500 mg.

61. The method of claim 60, wherein the Compound of Formula (A) or pharmaceutically acceptable salt thereof is administered at a dose of from about 5 to about 200 mg.

62. The method of any one of claims 44-61, wherein the atomoxetine or pharmaceutically acceptable salt thereof is administered at a dose of from about 20 to about 200 mg.

63. The method of claim 62, wherein the atomoxetine or pharmaceutically acceptable salt thereof is administered at a dose of from about 25 to about 100 mg.

64. The method of any one of claims 51-56, wherein the oxybutynin or pharmaceutically acceptable salt thereof is administered at a dose of from about 1 to about 15 mg.

65. The method of claim 64, wherein the oxybutynin or pharmaceutically acceptable salt thereof is administered at a dose of from about 2 mg to about 10 mg.

66. The method of any one of claims 51-55, wherein the (R)-oxybutynin or pharmaceutically acceptable salt thereof is administered at a dose of from about 0.5 to about 10 mg.

67. The method of claim 66, wherein the (R)-oxybutynin or pharmaceutically acceptable salt thereof is administered at a dose of from about 1 mg to about 5 mg.

68. The method of any one of claims 57-59, wherein the trazodone or pharmaceutically acceptable salt thereof is administered at a dose of from about 12.5 mg to about 200 mg.

69. The method of claim 44, 60 or 61 wherein the Compound of Formula (A) or a pharmaceutically acceptable salt thereof is administered as a monotherapy.

70. The method of any one of claims 45-68, wherein the NRI and the Compound of Formula (A) or a pharmaceutically acceptable salt thereof are administered in a single composition.

71. The method of any one of claims 51-56, wherein the NRI, MRA, and the Compound of Formula (A) or a pharmaceutically acceptable salt thereof are administered in a single composition.

72. The method of any one of claims 57-59, wherein the hypnotic and the Compound of Formula (A) or a pharmaceutically acceptable salt thereof are administered in a single composition.

73. The method of any one of claims 57-59, wherein the hypnotic, the NRI, and the Compound of Formula (A) or a pharmaceutically acceptable salt thereof are administered in a single composition.

74. The method of any one of claims 70-73, wherein the single composition is an oral administration form.

75. The method of claim 74, wherein the oral administration form is a syrup, pill, tablet, troche, capsule, or patch.

76. The method of any one of claims 44-75, wherein the condition associated with pharyngeal airway collapse is sleep apnea.

77. The method of claim 76, wherein the condition associated with pharyngeal airway collapse is obstructive sleep apnea (OSA).

78. The method of any one of claims 44-75, wherein the condition associated with pharyngeal airway collapse is snoring.

79. The method of claim 78, wherein the condition associated with pharyngeal airway collapse is simple snoring.

80. The method of any one of claims 44-79, wherein the subject is in a non-fully conscious state, such as sleep.

81. The method of any one of claims 44-80, wherein the subject has hypertension.

82. The method of claim 81, wherein the subject has OSA with hypertension.

83. The Compound of Formula (A) or a pharmaceutically acceptable salt thereof for use in treating obstructive sleep apnea.

84. The Compound of Formula (A) or a pharmaceutically acceptable salt thereof for use in treating obstructive sleep apnea with hypertension.

85. A therapeutic combination of the Compound of Formula (A) or a pharmaceutically acceptable salt thereof and (a) a norepinephrine reuptake inhibitor (NRI), and optionally a muscarinic receptor antagonist (MRA), or (b) a hypnotic, and optionally a norepinephrine reuptake inhibitor (NRI), for use in treating obstructive sleep apnea.

86. A therapeutic combination of the Compound of Formula (A) or a pharmaceutically acceptable salt thereof and (a) a norepinephrine reuptake inhibitor (NRI), optionally with a muscarinic receptor antagonist (MRA), or (b) a hypnotic, optionally with a norepinephrine reuptake inhibitor (NRI), for use in treating obstructive sleep apnea with hypertension.

87. A pharmaceutical composition comprising baxdrostat or a pharmaceutically acceptable salt thereof, a norepinephinephrine reuptake inhibitor, and a pharmaceutically acceptable carrier or excipient.

88. The pharmaceutical composition of claim 87, further comprising a hypnotic.

89. The pharmaceutical composition of claim 87, further comprising a muscarinic receptor antagonist (MRA).

90. A pharmaceutical composition comprising baxdrostat or a pharmaceutically acceptable salt thereof, a hypnotic, and a pharmaceutically acceptable carrier or excipient.

91. A pharmaceutical composition comprising the Compound of Formula (A) or a pharmaceutically acceptable salt thereof, a norepinephinephrine reuptake inhibitor, and a pharmaceutically acceptable carrier or excipient.

92. The pharmaceutical composition of claim 91, further comprising a hypnotic.

93. The pharmaceutical composition of claim 91, further comprising a muscarinic receptor antagonist (MRA).

94. A pharmaceutical composition comprising the Compound of Formula (A) or a pharmaceutically acceptable salt thereof, a hypnotic, and a pharmaceutically acceptable carrier or excipient.
